(19) United States
(12) Patent Application Publication
(10) Pub. No.: US 2008/0159961 A1
(43) Pub. Date: Jul. 3, 2008

(54) Pharmaceutical Composition

(76) Inventors: Austen John Woolfe, Essex (GB); Alan Keith Langford, Hertfordshire (GB); Jacqueline Yvonne Allen, London (GB); Mark Clifford Elliott, Stoke Newington (GB)

Correspondence Address: Martin A. Hay & CO., White Cottage . 33 Castle Hill, Prestbury / Cheshire, SK10 4AS, omitted

(21) Appl. No.: 10/516,943

(22) PCT Filed: Jun. 20, 2003
(86) PCT No.: PCT/GB2003/002669

§ 371 Date: Nov. 17, 2005

(30) Foreign Application Priority Data
Jun. 22, 2002 (US) 0214491.3
Dec. 3, 2002 (US) 0228111.1

(51) Int. Cl. A61K 9/12

(52) U.S. Cl. 424/45

(57) ABSTRACT

A pharmaceutical composition for administration as an aerosol, which comprises a cannabinoid, a propellant and an effective amount of a cough suppressant.

The present invention relates to a pharmaceutical composition. More particularly, it relates to an aerosol composition comprising a cannabinoid, to a metered dose dispenser containing the composition and to a method of administering the composition to a patient.

Cannabis is known to be useful in therapy, for example in the treatment of nausea and vomiting associated with cancer chemotherapy, anorexia associated with AIDS, pain, epilepsy, glaucoma, asthma and mood disorders. The principle active ingredient in cannabis is delta-9-tetrahydrocannabinol (delta-9-THC). A derivative of delta-9-THC, which possesses similar properties, is delta-8-tetrahydrocannabinol (delta-8-THC). Collectively, cannabis, delta-9-THC and derivatives thereof, such as delta-8-THC, are known as cannabinoids.

International patent application publication number WO 01/66089 and United States patent application publication number 2002/0031480 disclose aerosol compositions comprising a cannabinoid and a propellant for administration to patients using a metered dose dispenser. WO 03/006010, published on 23 Jan. 2003, also discloses aerosol compositions comprising a cannabinoid and a propellant for administration to patients using a metered dose dispenser.

It is reported in WO 01/66089 that administration of aerosol compositions comprising the cannabinoid, delta-9-THC, and a propellant to the lungs of patients caused the patients to cough. Applicant has encountered a similar problem when administering aerosol formulations comprising delta-8-THC. This cough reaction is undesirable, because it results in exhalation of much of the inhaled dose.

Surprisingly, it has now been found that by incorporating a sufficient amount of a certain kind of ingredient into the aerosol compositions, the cough reaction of patients is suppressed.

According to one aspect, therefore, the present invention provides a pharmaceutical composition for administration as an aerosol, which comprises a cannabinoid, a propellant and an effective amount of a cough suppressant.

Particularly good results have been obtained by incorporating medium chain triglycerides and propylene glycol diesters in a weight ratio of triglyceride to cannabinoid of at least 2:1, with the best results being obtained using weight ratios of at least 3:1 together with ethanol as a co-solvent.

According to a preferred aspect, therefore the cough suppressant is a medium chain triglyceride or propylene glycol diester.

Medium chain triglycerides are well known in the pharmaceutical formulation art, where they are mainly used in oral, parenteral and topical formulations. They are generally commercially available as mixtures of triglycerides of fatty acids consisting predominantly of octanoic (caprylic) and decanoic (capric) acid and may thus be represented by the general formula

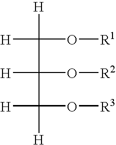

in which each of R¹, R² and R³ independently represents a group of formula —CO—(CH₂)ₙ—CH₃ in which n is an integer of from 6 to 8.

Examples of commercially available medium chain triglycerides are MIGLYOL™ 810 and 812, both caprylic/capric triglycerides available from CONDEA Chemie GmbH, Oleochemicals, Arthur-Imhausen-Str. 92, D-58433 Witten, Germany or CONDEA Vista Co., Commerce Dr., Cranford, N.J. 07016, United States, and CRODAMOL™ GTCC or CRODAMOL™ PC DAB 10(S), both caprylic/capric triglycerides, available from Croda chemicals Ltd., Rawcliffe Bridge, Goole, East Riding, DN14 8PN.

Medium chain diesters of propylene glycol are generally commercially available as mixtures of diesters of fatty acids consisting predominantly of octanoic (caprylic) and decanoic (capric) acid and may thus be represented by the general formula

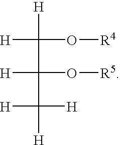

in which each of R⁴ and R⁵ independently represents a group of formula —CO—(CH₂)ₙ—CH₃ in which n is an integer of from 6 to 8.

An example of a commercially available medium chain diester of propylene glycol is MIGLYOL™ 840, a propylene glycol dicaprylate/dicaprate, available from CONDEA Chemie GmbH, Oleochemicals, Arthur-Imhausen-Str. 92, D-58433 Witten, Germany or CONDEA Vista Co., Commerce Dr., Cranford, N.J. 07016, United States.

The cough suppressant may conveniently be present in a weight ratio of cough suppressant to cannabinoid of from 2:1 to 25:1, preferably 2.5:1 to 15:1, most preferably 3:1 to 10:1.

The cannabinoid may be, for example, an extract of natural cannabis, delta-9-THC, a derivative of delta-9-THC such as delta-8-THC, cannabidiol, or a mixture of any of these. Preferably it is delta-8-THC.

The propellant may be, for example, an alkane, such as butane, or a fluorocarbon, such as 1,1,1,2-tetrafluoroethane (P-134a) or 1,1,1,2,3,3,3-heptafluoropropane (P-227). Preferably it is P-134a.

The weight ratio of propellant to cannabinoid in the composition is conveniently in the range of from 10:1 to 10,000:1, such as from 250:1 to 10,000:1, preferably from 50:1 to 500:1.

The composition may further comprise one or more solid or liquid carriers or excipients, such as a pharmaceutically acceptable solvent, for example an alcohol such as ethanol, an essential oil, such as peppermint, or a major component thereof, such as menthol, or a solid bulking agent, such as lactose. Preferably, the composition is a solution.

The one or more carriers or excipients in the aerosol composition may conveniently comprise from 0 to 25% by weight of the total composition.

It has been found to be advantageous to include ethanol in the composition. The ethanol may make up from 0.1% to 25% by weight of the formulation, preferably 1% to 25% of the formulation, more preferably 1% to 15%, most preferably from 3 to 5%. It has been found that when using high levels of ethanol, for example from 15 to 25% by weight, it is possible to use a lower ratio of cough suppressant to cannabinoid than is effective with low levels of ethanol. Furthermore, with high levels of ethanol, certain pharmaceutically acceptable aerosol surfactants, such as isopropyl myristate and Brij 30 (a lauryl polyoxyethylene ether), can function as cough suppressants. However, the best results have been obtained using medium chain triglycerides and propylene glycol diesters in compositions containing from 3 to 5% by weight ethanol.

In certain cases, administration of the cannabinoid has been found to be associated with undesirable after effects, such as a burning or tingling sensation in the throat, or a dry throat. It has been found that these effects may be reduced or eliminated by incorporating an essential oil in the composition. Examples of essential oils include peppermint (of which the major constituent is menthol), eucalyptus (of which the major constituent is cineole), aniseed and cajeput. According to a preferred aspect, therefore, the composition according to the present invention may further comprise an essential oil, such as peppermint, eucalyptus, aniseed or cajeput, or a major component thereof, such as methanol or cineole. Particularly good results have been obtained by incorporating menthol in compositions. The essential oil (e.g. menthol) preferably comprises from 0.02 to 0.1% by weight of the composition. The weight ratio of essential oil to delta-8-THC is preferably in the range of from 0.05:1 to 0.4:1, more preferably 0.1:1 to 0.3:1.

The pharmaceutical composition according to the invention may conveniently be administered to a patient using a metered dose dispenser, such as a metered dose inhaler. According to another aspect, therefore, the present invention provides a metered dose dispenser containing a pharmaceutical composition according to the invention. Preferably the metered dose dispenser is adapted to provide a unit dose containing from 0.05 to 0.5 mg of the cannabinoid, preferably from 0.1 to 0.2 mg.

According to another aspect, the present invention provides a method of administering an aerosol composition comprising a cannabinoid and a propellant to a patient, which comprises administering the cannabinoid and propellant with an effective amount of a cough suppressant.

According to another aspect, the present invention provides the use of an effective amount of a cough suppressant in the manufacture of a medicament for suppressing coughing when an aerosol composition comprising a cannabinoid and a propellant is administered to a patient.

As used herein, the term patient refers to any human or non-human animal. Preferably the patient is a human.

The aerosol composition is conveniently administered by inhalation. However, it may be administered via a pulmonary, sub-lingual, nasal or buccal route. Thus, although the risk of provoking a cough is lower if an aerosol lacking a cough suppressant is administered via a sub-lingual, nasal or buccal route, it would be advantageous for patients to receive cannabinoid with a cough suppressant, in accordance with the present invention.

The following Examples illustrate the invention.

### EXAMPLE 1

| Ingredient | Weight in mg |
|---|---|
| delta-8-THC | 5.2 (0.1 mg dose) |
| P-134a | 1606 |
| Crodamol GTCC | 15.9 (3.1:1 cough suppressant:cannabinoid) |
| Ethanol | 42.7 (2.6% by weight) |

### COMPARISON EXAMPLE 1

| Ingredient | Weight in mg |
|---|---|
| delta-8-THC | 6.1 (0.12 mg) |
| P-134a | 1477 |
| Crodamol GTCC | 11.4 (1.9:1) |
| Ethanol | 50.1 (3.3%) |

Notes: A comparison between Example 1 and Comparison Example 1 shows that having a sufficient amount of Crodamol GTCC in the aerosol composition is important.

### EXAMPLE 2

| Ingredient | Weight in mg |
|---|---|
| delta-8-THC | 5.0 (0.12 mg) |
| P-134a | 1220 |
| Crodamol PC DAB 10(S) | 52 (10.4:1) |
| Ethanol | 0 (0%) |

### EXAMPLE 3

| Ingredient | Weight in mg |
|---|---|
| delta-8-THC | 5.0 (0.23 mg) |
| P-134a | 656 |
| Crodamol PC DAB 10(S) | 15.5 (3.1:1) |
| Ethanol | 49 (7%) |

### EXAMPLE 4

| Ingredient | Weight in mg |
|---|---|
| delta-8-THC | 5.1 (0.12 mg) |
| P-134a | 1288 |
| Crodamol PC DAB 10(S) | 15.1 (3:1) |
| Ethanol | 100 (7.2%) |

### EXAMPLE 5

| Ingredient | Weight in mg |
|---|---|
| delta-8-THC | 5.1 (0.12 mg) |
| P-134a | 1274 |
| Crodamol PC DAB 10(S) | 15.2 (3:1) |
| Ethanol | 45.9 (3.5%) |

### EXAMPLE 6

| Ingredient | Weight in mg |
|---|---|
| delta-8-THC | 5.2 (0.12 mg) |
| P-134a | 1301 |
| Crodamol PC DAB 10(S) | 16.8 (3.2:1) |
| Ethanol | 144.3 (10%) |

### EXAMPLE 7

| Ingredient | Weight in mg |
|---|---|
| delta-8-THC | 6 (0.15 mg) |
| P-134a | 1128 |
| Crodamol PC DAB 10(S) | 51 (8.5:1) |
| Ethanol | 64 (5.4%) |

### EXAMPLE 8

| Ingredient | Weight in mg |
|---|---|
| delta-8-THC | 10 (0.52 mg) |
| P-134a | 581 |
| Crodamol PC DAB 10(S) | 105 (10.5:1) |
| Ethanol | 0 (0%) |

### EXAMPLE 9

| Ingredient | Weight in mg |
|---|---|
| delta-8-THC | 20 (0.22 mg) |
| P-134a | 2689 |
| Crodamol PC DAB 10(S) | 300 (15:1) |
| Ethanol | 0 (0%) |

### COMPARISON EXAMPLE 2

| Ingredient | Weight in mg |
|---|---|
| delta-8-THC | 5 (0.24 mg) |
| P-134a | 634 |
| Crodamol PC DAB 10(S) | 5.5 (1.1:1) |
| Ethanol | 49 (7.2%) |

### COMPARISON EXAMPLE 3

| Ingredient | Weight in mg |
|---|---|
| delta-8-THC | 5.5 (0.13 mg) |
| P-134a | 1253 |
| Crodamol PC DAB 10(S) | 13.5 (2.5:1) |
| Ethanol | 101 (7.5%) |

### EXAMPLE 10

| Ingredient | Weight in mg |
|---|---|
| delta-8-THC | 10 (0.19 mg) |
| P-134a | 1340 |
| Crodamol PC DAB 10(S) | 58 (6.8:1) |
| Ethanol | 151 (10.1%) |
| Micronized lactose | 10 |

### EXAMPLE 11

| Ingredient | Weight in mg |
|---|---|
| delta-8-THC | 5.1 (0.12 mg) |
| P-134a | 1239 |
| Miglyol 810 | 17.7 (3.5:1) |
| Ethanol | 49.2 (3.8%) |

### EXAMPLE 12

| Ingredient | Weight in mg |
|---|---|
| delta-8-THC | 5.4 (0.09 mg) |
| P-134a | 1796 |
| Miglyol 812 | 18 (3.3:1) |
| Ethanol | 41.1 (2.2%) |

### EXAMPLE 13

| Ingredient | Weight in mg |
|---|---|
| delta-8-THC | 10 (0.09 mg) |
| P-134a | 3207 |
| Miglyol 812 | 20.8 (2.1:1) |
| Ethanol | 193.4 (5.7%) |

### EXAMPLE 14

| Ingredient | Weight in mg |
|---|---|
| delta-8-THC | 10 (0.1 mg) |
| P-134a | 3062 |
| Miglyol 812 | 20.3 (2:1) |
| Ethanol | 261.5 (7.9%) |

### COMPARISON EXAMPLE 4

| Ingredient | Weight in mg |
|---|---|
| delta-8-THC | 5.6 (0.09 mg) |
| P-134a | 1788 |
| Miglyol 812 | 12.3 (2.2:1) |
| Ethanol | 41.9 (2.3%) |

### COMPARISON EXAMPLE 5

| Ingredient | Weight in mg |
|---|---|
| delta-8-THC | 10.3 (0.1 mg) |
| P-134a | 3019 |
| Miglyol 840 | 20.8 (2:1) |
| Ethanol | 124.7 (4%) |

Notes: A comparison between Examples 13 and 14 and Comparison Examples 4 and 5 shows that increasing the percentage by weight of ethanol can compensate for a reduced cough suppressant/cannabinoid ratio.

### EXAMPLE 15

| Ingredient | Weight in mg |
|---|---|
| delta-8-THC | 25 (0.2 mg) |
| P-134a | 3451 |
| Miglyol 812 | 75 (3:1) |
| Ethanol | 145 (4%) |

### EXAMPLE 16

| Ingredient | Weight in mg |
|---|---|
| delta-8-THC | 52.4 (0.2 mg) |
| P-134a | 6952 |
| Miglyol 812 | 132.4 (2.5:1) |
| Ethanol | 597.9 (7.9%) |

### EXAMPLE 17

| Ingredient | Weight in mg |
|---|---|
| delta-8-THC | 6.6 (0.14 mg) |
| P-134a | 1423 |
| Miglyol 840 | 17.1 (2.6:1) |
| Ethanol | 48.6 (3.3%) |

### COMPARISON EXAMPLE 6

| Ingredient | Weight in mg |
|---|---|
| delta-8-THC | 4.97 (0.1 mg) |
| P-134a | 1137 |
| Ethanol | 274.2 (19.4%) |

### EXAMPLE 18

| Ingredient | Weight in mg |
|---|---|
| delta-8-THC | 25.4 (0.20 mg) |
| P-134a | 3568 |
| Miglyol 840 | 77.8 (3.1:1) |
| Ethanol | 146.18 (3.9%) |
| Eucalyptus Oil | 2.7 (0.07%) |

### EXAMPLE 19

| Ingredient | Weight in mg |
|---|---|
| delta-8-THC | 24.8 (0.20 mg) |
| P-134a | 3509 |
| Miglyol 840 | 78.4 (3.1:1) |
| Ethanol | 148.35 (4.1%) |
| Peppermint Oil | 2.7 (0.07%) |

### EXAMPLE 20

| Ingredient | Weight in mg |
|---|---|
| delta-8-THC | 12.46 (0.10 mg) |
| P-134a | 3500 |
| Miglyol 840 | 44.2 (3.5:1) |
| Ethanol | 145 (4.0%) |
| Menthol | 1.3 (0.04%, menthol:delta 8 0.1:1) |

### EXAMPLE 21

| Ingredient | Weight in mg |
|---|---|
| delta-8-THC | 5.0 (0.10 mg) |
| P-134a | 1380 |
| Miglyol 840 | 14.1 (2.8:1) |
| Ethanol | 63.2 (4.4%) |
| Menthol | 0.69 (0.05%, 0.14:1) |

### EXAMPLE 22

| Ingredient | Weight in mg |
|---|---|
| delta-8-THC | 2.6 (0.04 mg) |
| P-134a | 1861 |
| Miglyol 840 | 7.53 (2.9:1) |
| Ethanol | 62.7 (3.3%) |
| Menthol | 0.36 (0.02%, 0.14:1) |

### EXAMPLE 23

| Ingredient | Weight in mg |
|---|---|
| delta-8-THC | 2.62 (0.05 mg) |
| P-134a | 1512 |
| Miglyol 840 | 8.08 (3.1:1) |
| Ethanol | 62.1 (3.9%) |
| Menthol | 0.71 (0.04%, 0.27:1) |

### EXAMPLE 24

| Ingredient | Weight in mg |
|---|---|
| delta-8-THC | 5 (0.11 mg) |
| P-134a | 990 |
| Brij ™ 30 | 28 (5.5:1) |
| Ethanol | 249 (20%) |

### EXAMPLE 25

| Ingredient | Weight in mg |
|---|---|
| delta-8-THC | 6 (0.12 mg) |
| P-134a | 1068 |
| Isopropyl myristate | 31 (5:1) |
| Ethanol | 271 (20%) |

### EXAMPLE 26

| Ingredient | Weight in mg |
|---|---|
| delta-8-THC | 12 (0.1 mg/dose) |
| P-134a | 3430 |
| Miglyol 812 | 36 (3:1) |
| L-Menthol | 1.51 |
| Ethanol | 302 (8%) |

### EXAMPLE 27

| Ingredient | Weight in mg |
|---|---|
| delta-9-THC | 4.99 |
| P-134a | 1514.9 |
| Miglyol 812 | 17.38 |
| Ethanol | 63.7 |

### EXAMPLE 28

| Ingredient | Weight in mg |
|---|---|
| Cannabidiol | 11.9 |
| P-134a | 1814.0 |
| Miglyol 812 | 30.3 |
| Ethanol | 130.3 |

The effect of administering the compositions of the Examples and Comparison Examples on patients was investigated as follows:

The ingredients were filled in standard glass vials with a normal valve and seals. The completed units were put in a standard actuator and primed. Then one puff of each was taken in the normal manner by the volunteer.

The compositions of the Examples were found to produce no cough, whereas those of the Comparison Examples were found to produce a spontaneous cough within 2-3 seconds.

An experiment was also conducted to investigate whether the cough suppressant and cannabinoid could be administered sequentially. This is described below.

| | First Dose | Second Dose |
|---|---|---|
| Ingredient | Weight in mg | Weight in mg |
| delta-8-THC | 0 | 4.8 (0.01 mg) |
| P-134a | 1540.4 | 1502.0 |
| Miglyol 812 | 25.2 | |
| Ethanol | 65.4 (4.1%) | 62.3 (4.0%) |
| Eucalyptus Oil | 0 | 18.6 |

The first dose, containing Miglyol 812, was inhaled twice, then the second dose was inhaled. The ratio of Miglyol 812: delta-8-THC inhaled was 10.5:1. A spontaneous cough was provoked after 5 seconds. This experiment shows that the cough suppressant needs to be administered with the cannabinoid.

It will be understood that the Examples have been provided to illustrate the invention. The invention is not limited to compositions using the particular cough suppressants described in these Examples or particularly described herein. Following the teachings herein about how the cough reflex may be suppressed in aerosol formulations containing a cannabinoid and a propellant, those skilled in the art should readily be able to identify other cough suppressants.

1. A pharmaceutical composition for administration as an aerosol, which comprises a cannabinoid, a propellant and an effective amount of a cough suppressant.

2. A composition as claimed in claim 1, which is a solution.

3. A composition as claimed in claim 1, in which the weight ratio of cough suppressant to cannabinoid in the composition is in the range of from 2:1 to 25:1.

4. A composition as claimed in claim 3, in which the weight ratio of cough suppressant to cannabinoid in the composition is in the range of from 2.5:1 to 15:1.

5. A composition as claimed in claim 4, in which the weight ratio of cough suppressant to cannabinoid in the composition is in the range of from 3:1 to 10:1.

6. A composition as claimed in claim 1, in which the cough suppressant is a medium chain triglyceride or propylene glycol diester.

7. A composition as claimed in claim 1, in which the propellant is 1,1,1,2-tetrafluoroethane.

8. A composition as claimed in claim 1, in which the cannabinoid is delta-8-THC.

9. A composition as claimed in claim 1, which further comprises ethanol.

10. A composition as claimed in claim 9, which comprises from 1 to 15% by weight of ethanol.

11. A composition as claimed in claim 10, which comprises from 3 to 5% by weight of ethanol.

12. A composition as claimed in claim 1, which further comprises an essential oil or a major component thereof.

13. A composition as claimed in claim 12, in which the essential oil or a major component thereof is menthol.

14. A composition as claimed in claim 13, which comprises from 0.02 to 0.1% by weight of menthol.

15. A metered dose dispenser, which contains a pharmaceutical composition as claimed in claim 1.

16. A metered dose dispenser as claimed in claim 15, which is a metered dose inhaler.

17. A metered dose dispenser as claimed in claim 16, which is adapted to provide a unit dose containing from 0.1 to 0.2 mg of cannabinoid.

18. (canceled)

19. A method of administering an aerosol composition comprising a cannabinoid and a propellant to a patient, which comprises administering the cannabinoid and propellant with an effective amount of a cough suppressant.

20. A pharmaceutical composition for administration as an aerosol, which comprises a cannabinoid, a propellant and an amount of a medium chain triglyceride or propylene glycol diester effective as a cough suppressant.

21. A composition as claimed in claim 20, in which the cannabinoid is delta-8-THC.

22. A composition as claimed in claim 21, which comprises from 1 to 15% by weight of ethanol.

23. A composition as claimed in claim 22, which comprises from 3 to 5% by weight of ethanol.

24. A composition as claimed in claim 22, which further comprises an essential oil or a major component thereof.

25. A composition as claimed in claim 23, which further comprises from 0.02 to 0.1% by weight of menthol.

26. A method as claimed in claim 19, in which the cough suppressant is a medium chain triglyceride or propylene glycol diester.

27. A method as claimed in claim 26, in which the cannabinoid is delta-8-THC.
